(19) United States
(12) Patent Application Publication
(10) Pub. No.: US 2024/0221930 A1
(43) Pub. Date: Jul. 4, 2024

(54) ELECTRONIC DEVICE AND METHOD FOR DISPLAYING SCREEN ON BASIS OF ACQUIRED DATA

(71) Applicant: Samsung Electronics Co., Ltd., Suwon-si (KR)

(72) Inventors: Hyogil KIM, Suwon-si (KR); Intaek OH, Suwon-si (KR); Sung YOO, Suwon-si (KR); Donghyun LEE, Suwon-si (KR); Sunmin LEE, Suwon-si (KR); Sunghwan CHO, Suwon-si (KR)

(21) Appl. No.: 18/606,658

(22) Filed: Mar. 15, 2024

Related U.S. Application Data
(63) Continuation of application No. PCT/KR2022/012453, filed on Aug. 19, 2022.

(30) Foreign Application Priority Data
Sep. 29, 2021 (KR) 10-2021-0128408
Oct. 15, 2021 (KR) 10-2021-0137565

(51) Int. Cl. G16H 40/60; G16H 20/70

(52) U.S. Cl. CPC G16H 40/60; G16H 20/70

(57) ABSTRACT

An electronic device includes a display, at least one sensor, memory storing one or more computer programs and one or more processors communicatively coupled to the display, the at least one sensor and the memory. The one or more computer programs include computer-executable instructions that, when executed by the one or more processors, cause the electronic device to display a first visual object for guiding a first activity identified based on first data obtained from the electronic device in a first designated time interval, superimposed on a screen including time information in a second designated time interval, and, after displaying the first visual object in the second designated time interval, display a second visual object for guiding a second activity distinct from the first activity, identified based on second data obtained in a third designated time interval including the second designated time interval, superimposed on the screen.

### CROSS-REFERENCE TO RELATED APPLICATION(S)

This application is a continuation application, claiming priority under § 365(c), of an International application No. PCT/KR2022/012453, filed on Aug. 19, 2022, which is based on and claims the benefit of a Korean patent application number 10-2021-0128408, filed on Sep. 29, 2021, in the Korean Intellectual Property Office, and of a Korean patent application number 10-2021-0137565, filed on Oct. 15, 2021, in the Korean Intellectual Property Office, the disclosure of each of which is incorporated by reference herein in its entirety.

### BACKGROUND

### 1. Field

The disclosure relates to an electronic device and a method for displaying a screen based on acquired data.

### 2. Description of Related Art

Recently, as interest in health increases, various electronic devices for measuring a user's health conditions have been suggested, and various services for the user's health conditions are also provided.

Among the user's health conditions, a physical health condition may be easily recognized, while a psychological health condition is difficult to recognize. The proportion of diseases to psychological health is increasing in modern people who are exposed to various stresses. Therefore, measures to determine psychological health condition and induce treatment are being discussed.

The above information is presented as background information only to assist with an understanding of the disclosure. No determination has been made, and no assertion is made, as to whether any of the above might be applicable as prior art with regard to the disclosure.

### SUMMARY

In an electronic device, a method for determining a user's psychological health condition within a designated section and suggesting an activity recommended based on the determined result may be required.

In addition, when the activity recommended through the electronic device is suggested, it is necessary to identify whether the user's psychological health condition has improved. Therefore, even after the recommended activity is suggested, a method for continuously monitoring the user's psychological health condition may be required.

Aspects of the disclosure are to address at least the above-mentioned problems and/or disadvantages and to provide at least the advantages described below. Accordingly, an aspect of the disclosure is to provide an electronic device and a method for displaying a screen based on acquired data.

Additional aspects will be set forth in part in the description which follows and, in part, will be apparent from the description, or may be learned by practice of the presented embodiments.

In accordance with an aspect of the disclosure, an electronic device is provided. The electronic device includes a display, at least one sensor, memory storing one or more computer programs, and one or more processors operably communicatively coupled to the display, the at least one sensor and the memory, wherein the one or more computer programs include computer-executable instructions that, when executed by the one or more processors, cause the electronic device to display a first visual object for guiding a first activity identified based on first data obtained from the electronic device in a first designated time interval, superimposed on a screen including time information in a second designated time interval, and, after displaying the first visual object in the second designated time interval, display a second visual object for guiding a second activity distinct from the first activity, identified based on second data obtained in a third designated time interval including the second designated time interval, superimposed on the screen, wherein the first visual object is usable for executing a first application related to the first activity, and wherein the second visual object is usable for executing a second application related to the second activity, distinct from the first application.

In accordance with another aspect of the disclosure, a method performed by an electronic device is provided. The method includes displaying, by the electronic device, a first visual object for guiding a first activity identified based on first data obtained from the electronic device in a first designated time interval, superimposed on a screen including time information in a second designated time interval, and after displaying the first visual object in the second designated time interval, displaying, by the electronic device, a second visual object for guiding a second activity distinct from the first activity, identified based on second data obtained in a third designated time interval including the second designated time interval, superimposed on the screen, wherein the first visual object may be usable for executing a first application related to the first activity, and wherein the second visual object may be usable for executing a second application related to the second activity, distinct from the first application.

In accordance with another aspect of the disclosure, one or more non-transitory computer-readable storage media storing one or more programs including computer-executable instructions that, when executed by one or more processors of an electronic device including a display and at least one sensor, cause the electronic device to perform operations are provided. The operations include displaying, by the electronic device, a first visual object for guiding a first activity identified based on first data obtained from the electronic device in a first designated time interval, superimposed on a screen including time information in a second designated time interval, after displaying the first visual object in the second designated time interval, displaying, by the electronic device, a second visual object for guiding a second activity distinct from the first activity, identified based on second data obtained in a third designated time interval including the second designated time interval, superimposed on the screen, wherein the first visual object is usable for executing a first application related to the first activity, and wherein the second visual object is usable for executing a second application related to the second activity, distinct from the first application.

According to an embodiment, the electronic device displays a first visual object for guiding a first activity identified based on first data obtained within a first designated time interval, superimposed on a screen including time information in a second designated time interval. The electronic device displays a second visual object for guiding a second activity identified based on second data obtained within a third designated time interval including the second designated time interval, superimposed on the screen.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2 illustrates an environment including an electronic device according to an embodiment of the disclosure;

FIG. 3 is a simplified block diagram of an electronic device according to an embodiment of the disclosure;

FIG. 4 is a simplified block diagram of a processor included in an electronic device according to an embodiment of the disclosure;

FIG. 5 is a flowchart illustrating an operation of an electronic device according to an embodiment of the disclosure;

FIG. 6A illustrates a graph related to a stress level of a patient group and a normal group according to an embodiment of the disclosure;

FIG. 6B illustrates a graph related to a movement of a patient group and a normal group according to an embodiment of the disclosure;

FIG. 6C illustrates a graph related to walking of a patient group and a normal group according to an embodiment of the disclosure;

FIG. 6D illustrates a graph related to a sleep of a patient group and a normal group according to an embodiment of the disclosure;

FIG. 7 illustrates a first type to a fourth type classified by a first parameter and a second parameter according to an embodiment of the disclosure;

FIGS. 8A, 8B, 8C, 8D, and 8E illustrate an example of a screen displayed on an electronic device according to various embodiments of the disclosure;

FIG. 9 illustrates an example of designated time intervals according to an embodiment of the disclosure;

FIG. 10 is another flowchart illustrating an operation of an electronic device according to an embodiment of the disclosure;

FIG. 11 is another flowchart illustrating an operation of an electronic device according to an embodiment of the disclosure;

FIG. 12 illustrates another example of a screen displayed on an electronic device according to an embodiment of the disclosure;

FIG. 13 illustrates still another example of a screen displayed on an electronic device according to an embodiment of the disclosure;

FIG. 14A illustrates an example of a screen displayed on an electronic device and an external electronic device according to an embodiment of the disclosure;

FIG. 14B illustrates another example of a screen displayed on an electronic device and an external electronic device according to an embodiment of the disclosure;

FIG. 15 is another flowchart illustrating an operation of an electronic device according to an embodiment of the disclosure;

FIG. 16 illustrates still another example of a screen displayed on an electronic device according to an embodiment of the disclosure; and

FIG. 17 is still another flowchart illustrating an operation of an electronic device according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an integrated circuit (IC), or the like.

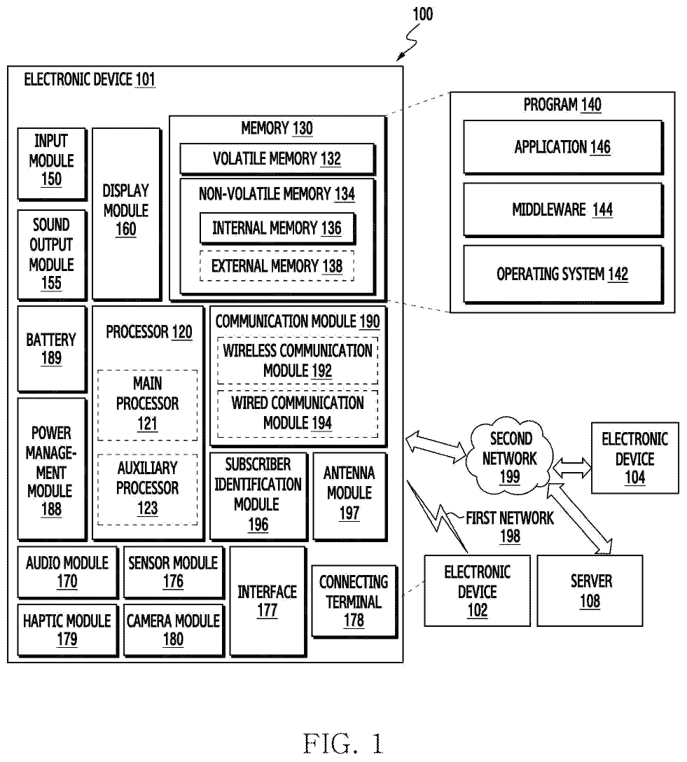

FIG. 1 is a block diagram illustrating an electronic device in a network environment according to an embodiment of the disclosure.

Referring to FIG. 1, an electronic device 101 in a network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 is adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102 or 104, or the server 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra-low-latency services using, e.g., distributed computing or mobile edge computing. In another embodiment, the external electronic device 104 may include an internet-of-things (IOT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

According to an embodiment, a processor (e.g., the processor 120 of FIG. 1) of an electronic device (e.g., the electronic device 101 of FIG. 1) may identify an activity to be proposed to a user of the electronic device based on data obtained within a designated time interval. The processor may display a first visual object for guiding the activity. After displaying the visual object, the processor may identify another activity based on data obtained within another designated time interval. The processor may display a second visual object for guiding the other activity.

An operation of a specific electronic device (or a processor of the electronic device) for the above-described embodiment may be described below. An electronic device described below may correspond to the electronic device 101 of FIG. 1.

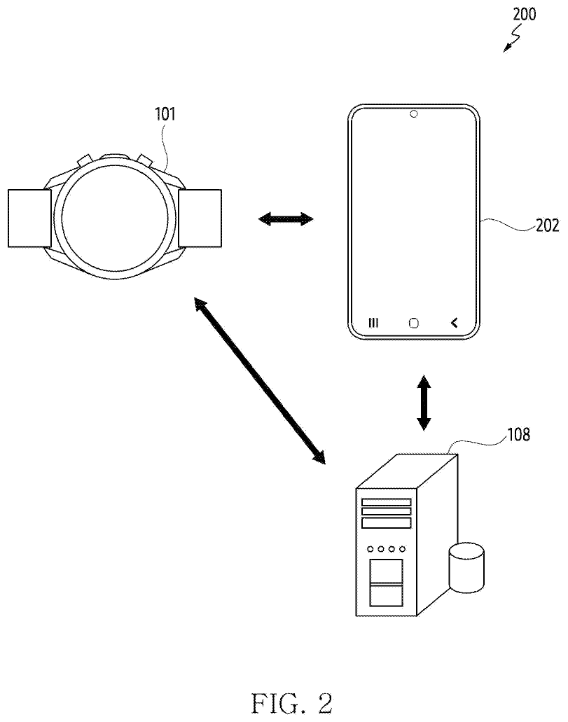

FIG. 2 illustrates an environment including an electronic device according to an embodiment of the disclosure.

Referring to FIG. 2, an environment 200 may include an electronic device 101, an external electronic device 202, and a server 108.

According to an embodiment, the electronic device 101 may operate while being worn by a user. For example, the electronic device 101 may operate by being worn on a part (e.g., a wrist) of the user's body. The electronic device 101 may have a watch shape.

According to an embodiment, the electronic device 101 may obtain data about the user. For example, the electronic device 101 may obtain physical data of the user. For example, the electronic device 101 may obtain at least one of data on a movement of the user and data on walking of the user. As another example, the electronic device 101 may obtain psychological data of the user. For example, the electronic device 101 may obtain at least one of data on a stress level of the user and data on sleep of the user.

According to an embodiment, the electronic device 101 may establish a connection with the external electronic device 202. For example, the external electronic device 202 may be used to control the electronic device 101. For example, the external electronic device 202 may transmit a request signal for controlling the electronic device 101 to the electronic device 101. The electronic device 101 may operate based on the request signal.

For example, the electronic device 101 may transmit information obtained through at least one sensor of the electronic device 101 to the external electronic device 202. The external electronic device 202 may process information received from the electronic device 101. The external electronic device 202 may identify an activity for guiding a user of the electronic device 101, based on the information received from the electronic device 101.

For example, the external electronic device 202 may receive information on the activity to be guided to the user from the electronic device 101. The external electronic device 202 may display information on the activity to be guided to the user on a lock screen.

According to an embodiment, the external electronic device 202 may obtain data on a body condition of the user through a user input. The external electronic device 202 may transmit the obtained data on the user's body condition to the electronic device 101.

According to an embodiment, the electronic device 101 may establish a connection with the server 108. For example, the server 108 may store information on a plurality of users including the user of the electronic device 101. For example, the server 108 may store information on a patient group for a designated disease (e.g., depression). The server 108 may transmit information on the patient group for the designated disease to the electronic device 101.

For example, the electronic device 101 may receive information for identifying data on the user's physical condition through a user input from the server 108. The electronic device 101 may obtain data on the user's physical condition through a user input, based on the received information.

For example, the electronic device 101 may be directly connected to the server 108 without passing through another device (e.g., the external electronic device 202). As another example, the electronic device 101 may be connected to the server 108 through the external electronic device 202.

Although FIG. 2 illustrates an example in which the electronic device 101 is connected and operated with the external electronic device 202, it is not limited thereto. The electronic device 101 may include at least a part of functions of the external electronic device 202. The electronic device 101 may operate independently of the external electronic device 202, by including at least a part of the functions of the external electronic device 202.

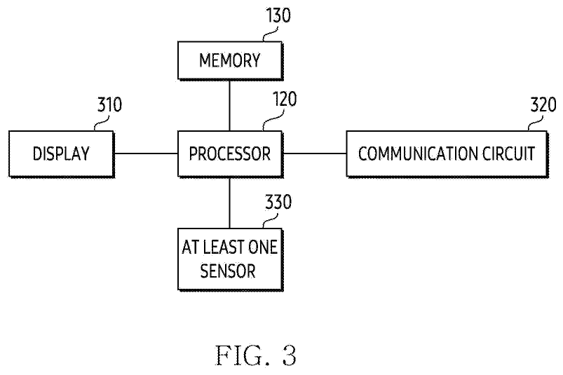

FIG. 3 is a simplified block diagram of an electronic device according to an embodiment of the disclosure.

Referring to FIG. 3, an electronic device 101 of FIG. 3 may correspond to the electronic device 101 of FIG. 1 and the electronic device 101 of FIG. 2. The electronic device 101 may include a processor 120, memory 130, a display 310, a communication circuit 320, and/or at least one sensor 330. According to an embodiment, the electronic device 101 may include at least one of the processor 120, the memory 130, the display 310, the communication circuit 320, and at least one sensor 330. For example, at least a part of the processor 120, the memory 130, the display 310, the communication circuit 320, and at least one sensor 330 may be omitted according to an embodiment.

According to an embodiment, the processor 120 may correspond to the processor 120 of FIG. 1. The processor 120 may be operably coupled with or connected with the memory 130, the display 310, the communication circuit 320, and at least one sensor 330.

According to an embodiment, the processor 120 may control the memory 130, the display 310, the communication circuit 320, and at least one sensor 330. The memory 130, the display 310, the communication circuit 320, and at least one sensor 330 may be controlled by the processor 120. For example, the processor 120 may obtain information stored in the memory 130. The processor 120 may identify information stored in the memory 130. For another example, the processor 120 may establish a connection with an external electronic device (e.g., the external electronic device 202 of FIG. 2) through the communication circuit 320.

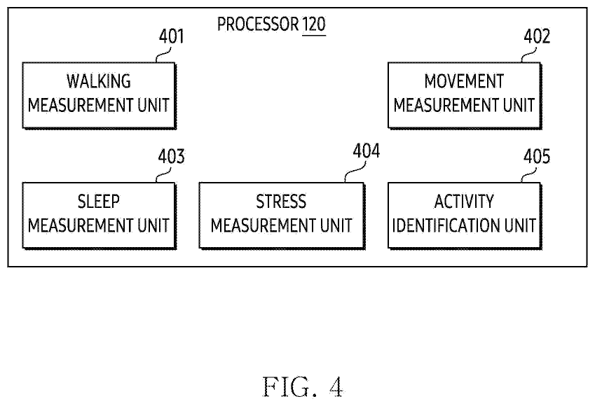

According to an embodiment, the processor 120 may identify data on a user and identify an activity based on the identified data. A specific configuration (or module) of the processor 120 for identifying the activity based on the identified data will be described later in FIG. 4.

According to an embodiment, the memory 130 may be used to store information or data. For example, the memory 130 may be used to store data obtained from the user. For example, the memory 130 may correspond to the memory 130 of FIG. 1. For example, the memory 130 may be a volatile memory unit or units. The memory 130 may be a nonvolatile memory unit or units. For another example, the memory 130 may be a computer-readable medium in another form, such as a magnetic or optical disk.

According to an embodiment, the display 310 may be used to display various screens. The display 310 may be used to output content, data, or signal through a screen. For example, the display 310 may correspond to the display module 160 of FIG. 1.

For example, the display 310 may display a screen processed by the processor 120. For example, the display 310 may display a screen including time information. The screen including time information may be processed (or generated) by the processor 120.

According to an embodiment, the communication circuit 320 may correspond to at least a part of the communication module 190 of FIG. 1. For example, the communication circuit 320 may be used for various radio access technologies (RATs). For example, the communication circuit 320 may be used to perform Bluetooth communication or wireless local area network (WLAN) communication. For another example, the communication circuit 320 may be used to perform cellular communication. For example, the processor 120 may establish a connection with an external electronic device (e.g., the external electronic device 202 of FIG. 2) through the communication circuit 320. For another example, the processor 120 may establish a connection with a server (e.g., the server 108 of FIG. 2) through the communication circuit 320.

According to an embodiment, the at least one sensor 330 may be used to obtain various external information. For example, the at least one sensor 330 may correspond to the sensor module 176 of FIG. 1.

For example, the at least one sensor 330 may include an acceleration sensor, a gyro sensor, or a magnetometer. The acceleration sensor may identify (or measure, or detect) acceleration of the electronic device 101 in three directions of the x-axis, y-axis, and z-axis. The gyro sensor may identify (or measure, or detect) an angular velocity of the electronic device 101 in three directions of the x-axis, y-axis, and z-axis. The magnetometer may detect magnitude of a magnetic field. For example, the magnetometer may be used to identify that the electronic device 101 is worn by the user based on a change in the magnitude of the magnetic field.

For example, the at least one sensor 330 may include a sensor for obtaining the user's biometric data. The processor 120 may detect at least one of blood pressure, electrocardiogram, heart rate variability (HRV), heart rate monitor (HRM), photoplethysmograph (PPG), sleep section, skin temperature, heart rate, blood flow, blood sugar, oxygen saturation, pulse wave, and electrocardiogram (ECG) through the at least one sensor 330. For example, the at least one sensor 330 may include a sensor for detecting at least one of blood pressure, electrocardiogram, heart rate variability (HRV), heart rate monitor (HRM), photoplethysmograph (PPG), sleep section, skin temperature, heart rate, blood flow, blood sugar, oxygen saturation, pulse wave, and electrocardiogram (ECG). As an example, the at least one sensor 330 may include a PPG sensor.

For example, the at least one sensor 330 may include an HRV sensor. The processor 120 may measure regularity or variability of heart rate through the HRV sensor. The processor 120 may obtain information on the regularity or variability of the heart rate through the HRV sensor. For example, the processor 120 may obtain information on variance of inter-beat interval (IBI) information between peak-to-peaks or information on deviations, based on heat rate (HR) information. The processor 120 may obtain information on heart rate regularity or variability, based on information on variance of IBI information or information on deviation. As another example, the processor 120 may obtain information on the regularity or variability of the heart rate, based on a frequency analysis of a heart rate signal.

For example, the at least one sensor 330 may include an electrode sensor. The processor 120 may identify (or measure) electrodermal activity (EDA) through the electrode sensor. The processor 120 may identify information on skin tension based on the EDA.

FIG. 4 is a simplified block diagram of a processor included in an electronic device according to an embodiment of the disclosure.

Referring to FIG. 4, a processor 120 of FIG. 4 may be an example of the processor 120 of the electronic device 101 of FIG. 3. According to an embodiment, the processor 120 may include a walking measurement unit 401, a movement measurement unit 402, a sleep measurement unit 403, a stress measurement unit 404, and/or an activity identification unit 405.

According to an embodiment, the walking measurement unit 401 may identify walking of a user, based on information on the impact amount identified through at least one sensor 330. For example, the walking measurement unit 401 may identify information on the impact amount or information on a step frequency. The walking measurement unit 401 may identify the user's walking state based on information on the impact amount or information on the step frequency. For example, the walking measurement unit 401 may identify whether the user is walking or running.

According to an embodiment, the walking measurement unit 401 may identify that a walking pattern is repeated within a designated section, in order to reduce measurement errors. The walking measurement unit 401 may identify that the user is walking based on identifying that the step pattern is repeated within the designated section.

According to an embodiment, the movement measurement unit 402 may identify a degree of movement of the user. Unlike the walking measurement unit 401 for identifying the designated walking pattern, the movement measurement unit 402 may identify a degree of simple movement of the user. For example, the movement measurement unit 402 may identify information on the number of movements per minute. For another example, the movement measurement unit 402 may identify at least one of a degree of movement, a persistence of movement, an intensity of movement, and/or a displacement difference of displacement difference, by using an inertial sensor among the at least one sensor 330.

According to an embodiment, the sleep measurement unit 403 may identify a sleep state of the user. For example, the sleep measurement unit 403 may identify a change in the user's biometric data through a PPG sensor among the at least one sensor 330. For example, the sleep measurement unit may monitor the user's biometric data through the PPG sensor. The sleep measurement unit 403 may identify the user's sleep state based on a change in the user's biometric data.

For example, the sleep measurement unit 403 may identify a heart rate signal of the user. The sleep measurement unit 403 may identify a sleep state (or sleep phase) based on a frequency analysis of the user's heart rate signal. For example, the sleep measurement unit 403 may identify the user's sleep state as one of a wake-up state, a state immediately before a hypnagogic state, a state immediately after a hypnagogic state, a light sleep state, a deep sleep state, and a rapid eye movement (REM) sleep state.

According to an embodiment, the stress measurement unit 404 may identify a stress level of the user. For example, the stress measurement unit 404 may identify the user's stress as one of acute stress and chronic stress.

For example, the stress measurement unit 404 may identify a stress level of the user based on data obtained through the HRV sensor among the at least one sensor 330. For another example, the stress measurement unit 404 may identify electrodermal activity (EDA) based on data obtained through an electrode sensor among the at least one sensor 330. The stress measurement unit 404 may identify the stress level of the user by identifying information on skin tension based on the EDA. For still another example, the stress measurement unit 404 may identify the user's stress level based on various biometric data (e.g., high resolution (HR)/HRV information or IBI information) of the users.

According to an embodiment, the activity identification unit 405 may identify an activity for guiding the user, based on data about the user. The activity identification unit 405 may display a visual object for guiding the identified activity on a screen through the display 310.

In the following descriptions, for convenience of explanation, operations performed in the walking measurement unit 401, the movement measurement unit 402, the sleep measurement unit 403, the stress measurement unit 404, and the activity identification unit 405 may be described as being performed by the processor 120.

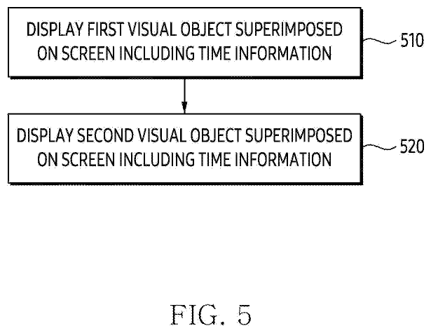

FIG. 5 is a flowchart illustrating an operation of an electronic device according to an embodiment of the disclosure.

This method may be executed by the electronic device 101 and the processor 120 of the electronic device 101 illustrated in FIGS. 2 and 3.

Referring to FIG. 5, in operation 510, the processor 120 may display a first visual object superimposed on a screen including time information. For example, the processor 120 may display the first visual object for guiding a first activity identified based on first data obtained within a first designated time interval, superimposed on the screen including the time information within a second designated time interval.

According to an embodiment, the processor 120 may obtain the first data within the first designated time interval (e.g., 1 week or 2 weeks). For example, the first data may include data about a user. The first data may include the user's physical data and the user's psychological data.

As an example, the user's physical data may include data on the user's movement, data on the user's walking, and/or data on the user's first sleep state (e.g., the deep sleep state). The processor 120 may identify one of data on the user's movement, data on the user's walking, and data on the user's first sleep state as the user's physical data.

As another example, the user's psychological data may include data on the user's stress level and/or data on the user's second sleep state (e.g., the REM sleep state). The processor 120 may identify at least one of data on the user's stress level and data on the user's second sleep state (e.g., the REM sleep state) as the user's psychological data.

According to an embodiment, the processor 120 may identify the first activity based on the first data. For example, the processor 120 may identify one of a first type to a fourth type based on the user's physical data and the user's psychological data. The first type to the fourth type may be used to classify a plurality of activities.

As an example, the processor 120 may identify a first parameter based on the user's psychological data. The processor 120 may identify a second parameter based on the user's physical data. The first parameter may be set to one of a first value (e.g., true or 1) and a second value (e.g., false or 0). The second parameter may be set to one of a first value (e.g., true or 1) and a second value (e.g., false or 0).

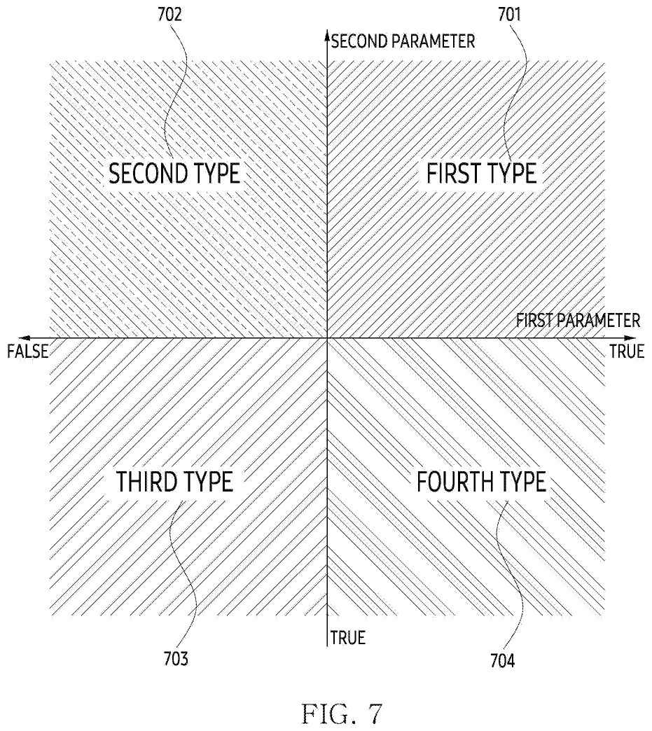

The processor 120 may identify one of the first type to the fourth type based on the first parameter and the second parameter. The processor 120 may identify one of a plurality of activities for the identified type as the first activity. A specific description of the first type to the fourth type will be described later in FIG. 7.

According to an embodiment, the processor 120 may display the first visual object for guiding the first activity, superimposed on a screen including time information. For example, the screen including time information may include a standby screen, a lock screen, and/or a watch face of the electronic device 101.

For example, the first visual object may be usable to execute a first application related to the first activity. The processor 120 may identify a user input with respect to the first visual object. The user input with respect to the first visual object may include a single tap input, a double tap input, a drag input, and a swipe input. The processor 120 may execute the first application based on the user input.

For example, the first activity may be identified as a phone call. The processor 120 may display a first visual object for executing a phone call application related to a phone call. For another example, the first activity may be identified as music listening. The processor 120 may display a first visual object for executing a music playback application related to music listening.

According to an embodiment, the electronic device 101 may operate in connection with the external electronic device 202. The first application of the electronic device 101 may be executed by being linked with the external electronic device 202. The processor 120 may transmit a signal for executing the first application to the external electronic device 202 connected with the electronic device 101, in response to a user input. The external electronic device 202 may execute the first application based on a received signal.

According to an embodiment, the processor 120 may display the first visual object within a second designated time interval (e.g., 1 day or 3 day). For example, the processor 120 may display the first visual object within the second designated time interval by a designated cycle. As an example, the processor 120 may display the first visual object at a designated time (e.g., 2 p.m.) within the second designated time interval. As another example, the processor 120 may display the first visual object within the second designated time interval, by repeating a first section in which the first visual object is displayed and a second section in which the second visual object is not displayed.

For another example, after a state of the display 310 of the electronic device 101 is changed from an inactive state to an active state, the processor 120 may display the first visual object, superimposed on a screen including time information. The processor 120 may remove the first visual object after a certain time elapses. The first visual object may be removed after a certain time elapses from a timing superimposed a screen including time information. The first visual object may disappear after a certain time elapses from the timing superimposed on a screen including time information.

According to an embodiment, the processor 120 may display a third visual object including text for proposing use of the first application, superimposed on a screen including time information together with the second visual object.

In operation 520, the processor 120 may display the second visual object, superimposed on a screen including time information. The processor 120 may display a second visual object for guiding a second activity distinct from the first activity, identified based on second data obtained within a third designated time interval including the second designated time interval, superimposed on a screen including time information.

According to an embodiment, the processor 120 may obtain the second data within the third designated time interval including the second designated time interval. For example, the second data may be related to the first data. For example, the second data may include data about the user. The second data may include physical data of the user and psychological data of the user.

For example, the second visual object may be used to execute a second application related to the second activity and distinct from the first application. The processor 120 may identify a user input with respect to the second visual object. The processor 120 may execute the second application based on the user input.

According to an embodiment, the processor 120 may identify the second activity based on the second data. The processor 120 may display a second visual object for guiding the identified second activity, superimposed on a screen including time information. An operation of displaying the second visual object may be performed in the same as or similar to an operation of displaying the first visual object.

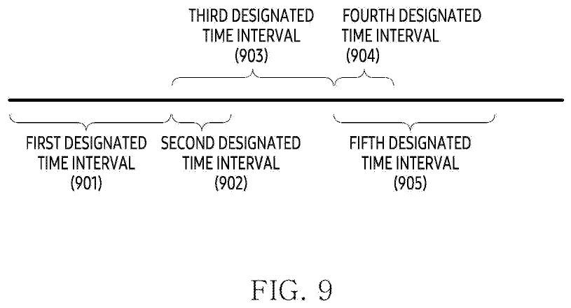

According to an embodiment, the first designated time interval to the third designated time interval described above may be variously set. For example, the first designated time interval to the third designated time interval may be set based on a user input. For another example, the first designated time interval to the third designated time interval may be set through the server 108. The processor 120 may receive information on the first designated time interval to the third designated time interval from the server 108. A specific descriptions of the first designated time interval to the third designated time interval will be described later in FIG. 9.

According to an embodiment, a visual object (e.g., the first visual object or the second visual object) displayed on the electronic device 101 may be referred to in various ways. For example, the visual object displayed on the electronic device 101 may be referred to as a complication. For example, the visual object displayed on the electronic device 101 may be set and changed by a user. As another example, the visual object displayed on the device 101 may be changed based on a first parameter and a second parameter described below.

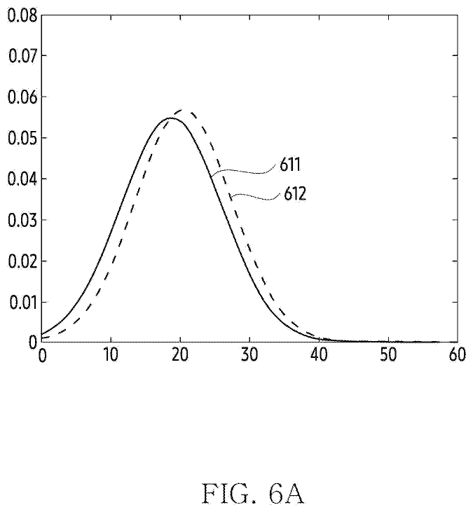

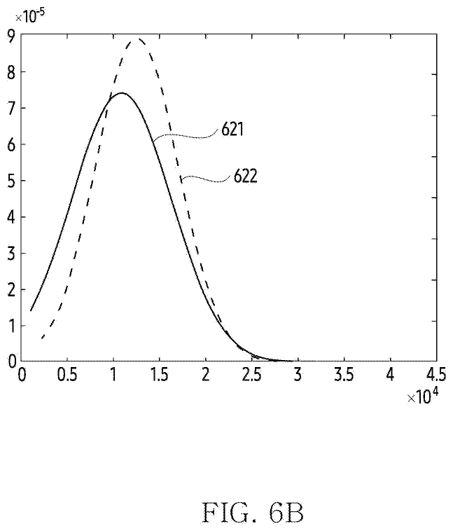

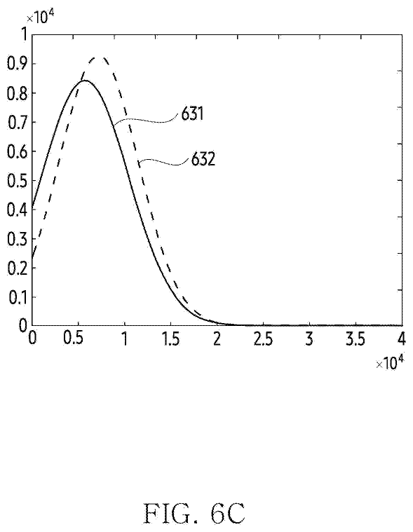

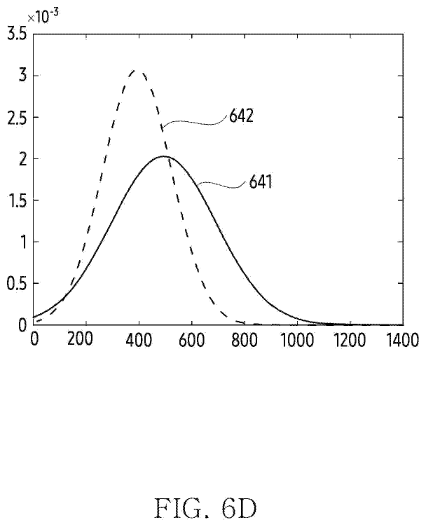

FIG. 6A illustrates a graph related to a stress level of a patient group and a normal group according to an embodiment of the disclosure.

FIG. 6B illustrates a graph related to a movement of a patient group and a normal group according to an embodiment of the disclosure.

FIG. 6C illustrates a graph related to walking of a patient group and a normal group according to an embodiment of the disclosure.

FIG. 6D illustrates a graph related to a sleep of a patient group and a normal group according to an embodiment of the disclosure.

Referring to FIGS. 6A to 6D, a processor 120 may identify data on a patient group and a normal group for a designated disease (e.g., depression). The processor 120 may receive data on a patient group and a normal group for a designated disease from the server 108. The processor 120 may obtain first data within a first time interval, based on the data on a patient group and a normal group for a designated disease.

Referring to FIG. 6A, a graph 611 and a graph 612 may be formed of a probability density function. The x-axis of the graph 611 and the graph 612 is ratio values of medium stress during a day. The y-axis of the graph 611 and the graph 612 are probability values.

The graph 611 illustrates a probability with respect to a ratio of medium stress during a day in a patient group for a designated disease (e.g., depression). The graph 612 illustrates a probability with respect to a ratio of medium stress during a day in a normal group. Comparing the graph 611 and the graph 612, in a case of the patient group, the percentage of medium stress during a day is lower than the normal group. For example, the proportion of medium stress during a day in the patient group is 90% of the normal group. Therefore, a stress level of the patient group is distributed to low stress or high stress.

Referring to FIG. 6B, a graph 621 and a graph 622 may include a probability density function. The x-axis of the graph 621 and the graph 622 is the number of movements per minute. The y-axis of the graph 621 and the graph 622 are probability values.

The graph 621 illustrates a probability with respect to the number of movements per minute in the patient group for a designated disease (e.g., depression). The graph 622 illustrates a probability with respect to the number of movements per minute in the normal group. Comparing the graph 621 with the graph 622, in a case of the patient group, the average number of movements per minute is lower than the normal group.

Referring to FIG. 6C, a graph 631 and a graph 632 may be formed of a probability density function. The x-axis of the graph 631 and the graph 632 is the number of steps during a day. The y-axis of the graph 631 and the graph 632 are probability values.

The graph 631 illustrates a probability with respect to the number of steps during a day in a patient group for a designated disease (e.g., depression). The graph 632 illustrates a probability for the number of steps during a day in the normal group. Comparing the graph 631 with the graph 632, in a case of the patient group, the average number of movements per minute is lower than the normal group.

Referring to FIG. 6D, a graph 641 and a graph 642 may comprise a probability density function. The x-axis of the graph 641 and the graph 642 are sleep times. The y-axis of the graph 641 and the graph 642 are probability values.

The graph 641 illustrates a probability of a sleep time in a patient group for a designated disease (e.g., depression). The graph 642 illustrates a probability of a sleep time in a normal group. Comparing the graph 641 and the graph 642, the average sleep time is long in a case of the patient group. The distribution of a difference between the sleep time in the patient group and the average sleep time (e.g., 7 hours) in the normal group is largely distributed. In other words, the variance of the patient group's sleep time is greater than the variance of the normal group's sleep time.

FIG. 7 illustrates a first type to a fourth type classified by a first parameter and a second parameter according to an embodiment of the disclosure.

Referring to FIG. 7, a plurality of activities may be classified into a first type 701 to a fourth type 704. For example, a plurality of activities may include candidates of the activity that may be identified to guide the user.

According to an embodiment, a processor 120 may identify a first activity based on first data. For example, the processor 120 may identify one of the first type 701 to the fourth type 704 based on the user's physical data and the user's psychological data.

The processor 120 may identify a first parameter based on the user's psychological data. The processor 120 may identify a second parameter based on the user's physical data. The first parameter may be set to one of a first value (e.g., true) and a second value (e.g., false). The second parameter may be set to one of the first value (e.g., true) and the second value (e.g., false).

The processor 120 may identify one of the first type 701 to the fourth type 704 based on the first parameter and the second parameter. The processor 120 may identify one of a plurality of activities for the identified type as the first activity.

According to an embodiment, the processor 120 may obtain the user's psychological data within a first designated time interval (e.g., 2 weeks). For example, the processor 120 may identify the first parameter based on the user's psychological data. The processor 120 may identify whether a designated condition is satisfied, based on the user's psychological data. The processor 120 may identify the first parameter, based on whether the designated condition is satisfied.

For example, the processor 120 may identify whether a ratio of the medium stress of the user during a day is less than or equal to a designated ratio (e.g., 18%). The processor 120 may identify whether a ratio of the medium stress during the first designated interval of the user during a day is less than or equal to the designated ratio (e.g., 18%). The processor 120 may identify that a designated condition is satisfied, based on identifying that the number of days in which the ratio of the medium stress during the first designated time interval is less than or equal to the designated ratio (e.g., 18%) is greater than or equal to a designated value. The processor 120 may identify the first parameter as the second value based on identifying that the designated condition is satisfied. The processor 120 may identify the first parameter as the first value based on identifying that the designated condition is not satisfied.

As another example, the processor 120 may identify data on a second sleep state (e.g., the REM sleep state) of the user. The processor 120 may identify whether a timing of entering the second sleep state from a timing of entering a sleep state is less than or equal to a certain time (e.g., 60 minutes). The processor 120 may identify that a designated condition is satisfied, based on identifying that the number of days in which the timing of entering the second sleep state from the timing of entering the sleep state during the first designated time interval is less than or equal to a certain time (e.g., 60 minutes) is greater than or equal to a designated value. The processor 120 may identify the first parameter as the second value, based on identifying that the designated condition is satisfied. The processor 120 may identify the first parameter as the first value, based on identifying that the designated condition is not satisfied.

As described above, as an example, an operation of identifying the first parameter based on the ratio of medium stress and an operation of identifying the first parameter based on data about the second sleep state are, and are not limited thereto. The processor 120 may identify the first parameter as one of the first value and the second value based on at least one condition variously set.

According to an embodiment, the processor 120 may obtain the user's physical data within the first designated time interval (e.g., 2 weeks). For example, the processor 120 identifies the second parameter based on the user's physical data. The processor 120 may identify whether the designated condition is satisfied, based on the user's physical data. The processor 120 may identify the second parameter, based on whether the designated condition is satisfied.

For example, the processor 120 identifies that the average number of the user's steps during a day is less than the number of designated steps (e.g., 5000 steps). The processor 120 may identify that a designated condition is satisfied, based on identifying that the number of days in which the average number of the user's steps during a day is less than the number of the designated steps is greater than or equal to a designated value during the first designated time interval. The processor 120 may identify the second parameter as the second value, based on identifying that the designated condition is satisfied. The processor 120 may identify the second parameter as the first value based on identifying that the designated condition is not satisfied.

As another example, the processor 120 may identify that the average number of the user's movements during a day is less than the number of designated movements (e.g., 10,000 times). The processor 120 may identify that a designated condition is satisfied, based on identifying that the number of days in which the average number of the user's movements during a day is less than the number of designated movements is greater than or equal to a designated value during the first designated time interval. The processor 120 may identify the second parameter as the second value based on identifying that the designated condition is satisfied. The processor 120 may identify the second parameter as the first value based on identifying that the designated condition is not satisfied.

As still another example, the processor 120 may identify data on the user's first sleep state (e.g., a deep sleep state). The processor 120 may identify whether a ratio of a time maintaining the first sleep state with respect to the user's sleep time is less than a designated ratio (e.g., 5%). The processor 120 may identify that a designated condition is satisfied, based on identifying that the ratio of the time maintaining the first sleep state with respect to the user's sleep time is less than the designated ratio during the first designated time interval. The processor 120 may identify the second parameter as the second value based on identifying that the designated condition is satisfied. The processor 120 may identify the second parameter as the first value based on identifying that the designated condition is not satisfied.

As described above, the operation of identifying the second parameter based on the average number of steps during a day, the operation of identifying the second parameter based on the average number of movements during a day, and the operation of identifying the second parameter based on the ratio of a time maintaining the first sleep state with respect to the sleep time are examples of disclosed aspects, and are not limited thereto. The processor 120 may identify the second parameter as one of the first value and the second value based on at least one condition variously set.

According to an embodiment, the processor 120 may identify the first type 701 among the first type 701 to the fourth type 704, based on the first parameter being identified as a first value (e.g., true) and the second parameter being identified as a second value (e.g., false). A plurality of activities related to the first type 701 may include a light exercise or stretching operation. The processor 120 may identify one of the plurality of activities related to the first type 701 as the first activity.

According to an embodiment, the processor 120 may identify the second type 702 among the first type 701 to the fourth type 704, based on the first parameter being identified as the second value (e.g., false) and the second parameter being identified as the second value (e.g., false). The plurality of activities related to the second type 702 may include a phone call or counseling. The processor 120 may identify one of the plurality of activities related to the second type 702 as the first activity.

According to an embodiment, the processor 120 may identify the third type 703 among the first type 701 to the fourth type 704 based on the first parameter being identified as the second value (e.g., false) and the second parameter being identified as the first value (e.g., true). The plurality of activities related to the third type 703 may include a breathing exercise or meditations. The processor 120 may identify one of the plurality of activities related to the third type 703 as the first activity.

According to an embodiment, the processor 120 may identify the fourth type 704 among the first type 701 to the fourth type 704 based on the first parameter being identified as the first value (e.g., true) and the second parameter being identified as the first value (e.g., true). The plurality of activities related to the fourth type 704 may include gameplay or video/music appreciation. The processor 120 may identify one of the plurality of activities related to the fourth type 704 as the first activity.

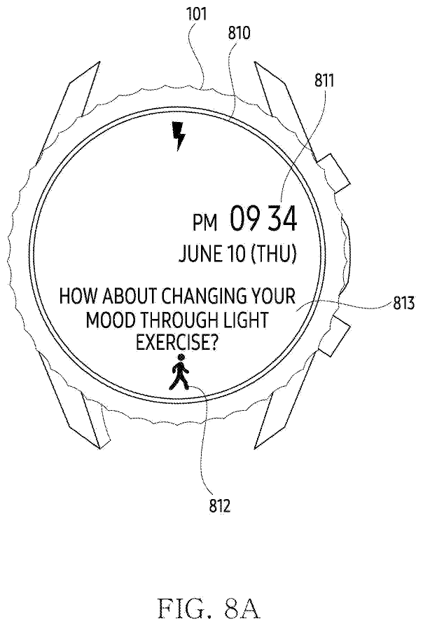

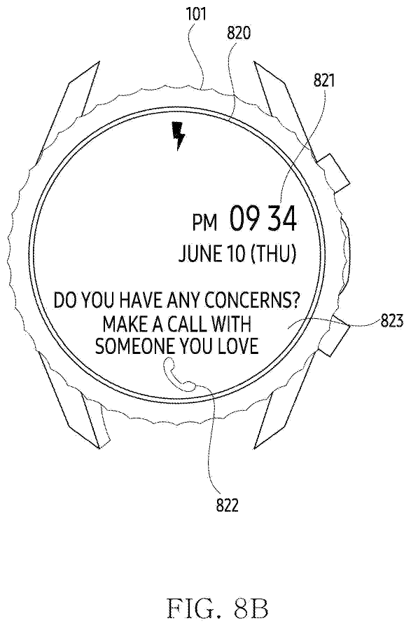

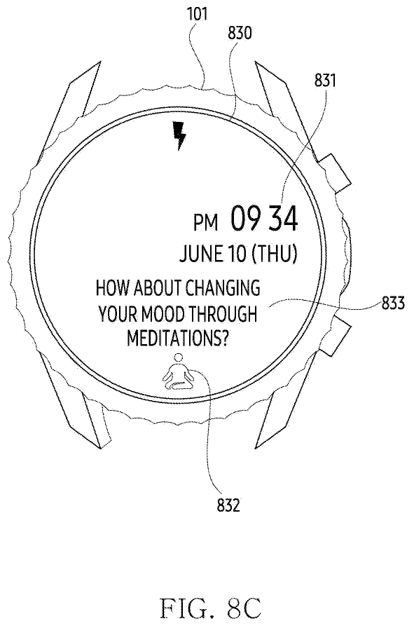

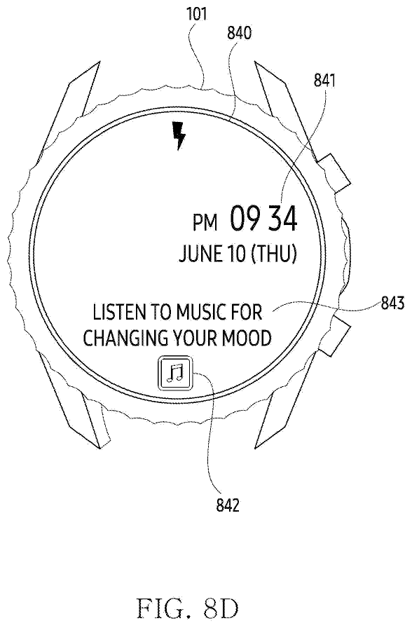

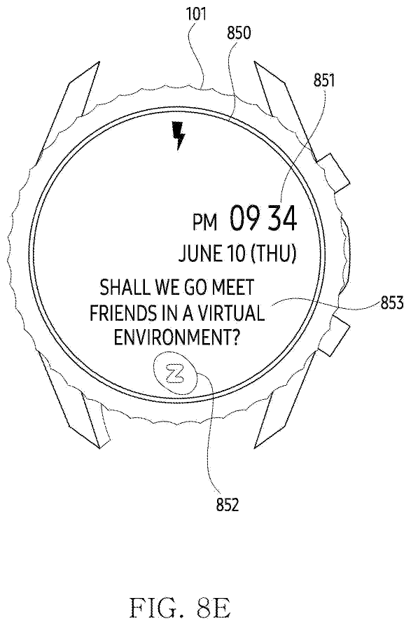

According to an embodiment, the first type 701 to the fourth type 704 may be set to guide different activities according to a user's state (or depression state). For example, when the user's state (or depression state) is serious, the processor 120 identifies the second type. The processor 120 may identify one of a plurality of activities (e.g., a phone call or counseling) related to the second type as the first activity. An example of an operation of the electronic device 101 for guiding different activities according to the user's state may be described in FIGS. 8A to 8E.

FIGS. 8A, 8B, 8C, 8D, and 8E illustrate an example of a screen displayed on an electronic device according to various embodiments of the disclosure.

Referring to FIGS. 8A to 8E, the processor 120 of the electronic device 101 may display a visual object (e.g., a first visual object or a second visual object) for guiding the first activity, superimposed on a screen including time information. According to an embodiment, the processor 120 may display a visual object including text for suggesting the use of an application (e.g., a first application or a second application), superimposed on a screen including time information together with a visual object for guiding the first activity.

For example, the processor 120 identifies the first parameter and the second parameter described in FIG. 7. The processor 120 may display a visual object for guiding the first activity to the user, superimposed on a screen including time information, based on the first parameter and the second parameter. For example, the processor 120 identifies the user's state (or depression state) based on the first parameter and the second parameter. The processor 120 may identify the first activity based on the user's state and display a visual object for guiding the first activity, superimposed on a screen including time information. In FIGS. 8A to 8E, an example of displaying a visual object for guiding the first activity identified based on the user's state may be illustrated.

Referring to FIG. 8A, the processor 120 may display a screen 810. The screen 810 may include time information. For example, the screen 810 displays a visual object 811 indicating time information. The processor 120 may identify the first type among the first type to the fourth type, based on the first parameter and the second parameter. The processor 120 may identify a light exercise among a plurality of activities related to the first type as the first activity. The processor 120 may display a visual object 812 for guiding the light exercise and a visual object 813 including text for suggesting the light exercise, superimposed on the screen 810.

Referring to FIG. 8B, the processor 120 may display a screen 820. The screen 820 may include time information. For example, the screen 820 includes a visual object 821 indicating time information. The processor 120 may identify a second type among the first to fourth types based on the first parameter and the second parameter. The processor 120 may identify a phone call among a plurality of activities related to the second type as the first activity. The processor 120 may display a visual object 822 for guiding a phone call and a visual object 823 including text for suggesting a phone call, superimposed on the screen 820.

For example, the processor 120 identifies a user input with respect to the visual object 822. The processor 120 may execute an application for a phone call in response to the user input with respect to the visual object 822.

For example, the processor 120 performs a phone call connection to a designated target in response to the user input with respect to the visual object 822. The processor 120 may display the visual object 823 including text for suggesting a phone call with the designated target. The designated target may include a counselling center, family, friend, or artificial intelligence (AI)-based counselor.

Referring to FIG. 8C, the processor 120 may display a screen 830. The screen 830 may include time information. For example, the screen 830 includes a visual object 831 indicating time information. The processor 120 may identify a third type among the first to fourth types based on the first parameter and the second parameter. The processor 120 may identify meditations or a breathing exercise among a plurality of activities related to the third type as the first activity. The processor 120 may display a visual object 832 for guiding meditations and a visual object 833 including text for suggesting meditations, superimposed on the screen 830.

According to an embodiment, the visual object 832 may be used to execute an application for guiding the user's breathing. For example, the processor 120 executes an application for guiding breathing in response to a user input to the visual object 832. According to an embodiment, the visual object 832 may be used to perform (or execute) a function performed in a designated application (e.g., an AI application or a health related application). For example, the processor 120 executes a designated application and perform a function performed within the designated application, in response to the user input to the visual object 832.

Referring to FIG. 8D, the processor 120 may display a screen 840. The screen 840 may include time information. For example, the screen 840 includes a visual object 841 indicating time information. The processor 120 may identify a fourth type among the first to fourth types based on the first parameter and the second parameter. The processor 120 may identify a media contents application among a plurality of activities related to the fourth type as the first activity. The processor 120 may display a visual object 842 for guiding music appreciation and a visual object 843 including text for suggesting music appreciation, superimposed on the screen 840.

For example, the processor 120 identifies a user input to the visual object 842. The processor 120 may execute an application for playing music in response to the user input to the visual object 842.

According to an embodiment, the processor 120 may identify information on a user's mood. The processor 120 may identify (or recommend) an application based on information on the user's mood. For example, the processor 120 displays the visual object 842 for guiding music appreciation for mood change, based on the information on the user's mood. For another example, the processor 120 may display the visual object 842 for listening to a media broadcast (e.g., podcast).

Referring to FIG. 8E, the processor 120 may display a screen 850. The screen 850 may include time information. For example, the screen 850 includes a visual object 851 indicating time information. The processor 120 may identify the fourth type among the first to fourth types based on the first parameter and the second parameter. The processor 120 may identify an activity in a virtual environment among the plurality of activities related to the fourth type as the first activity. The processor 120 may display a visual object 852 for guiding an activity in a virtual environment (or metaverse), and a visual object 853 including text for suggesting the activity in the virtual environment, superimposed on the screen 850.

For example, the processor 120 identifies a user input with respect to the visual object 852. The processor 120 may execute an application for an activity in a virtual environment in response to the user input to the visual object 852. According to an embodiment, the processor 120 may identify a second type among the first to fourth types based on the first parameter and the second parameter. The processor 120 may identify a counseling service in the virtual environment among a plurality of activities related to the second type as the first activity. The processor 120 may display the visual object 852 for guiding to perform a counseling service with an AI counselor in the virtual environment and a visual object including text for suggesting the counseling service in the virtual environment, superimposed on the screen 850.

FIG. 9 illustrates an example of designated time intervals according to an embodiment of the disclosure.

Referring to FIG. 9, the processor 120 of the electronic device 101 may identify a first activity based on first data obtained within a first designated time interval 901. For example, in the first designated time interval 901, the processor 120 obtains the user's physical data and the user's psychological data included in the first data. For example, the first designated time interval 901 is set to 2 weeks.

According to an embodiment, after the first designated time elapses, the processor 120 may identify data on the user's physical condition (hereinafter, referred to as third data) based on a user input (hereinafter, referred to as a first user input). For example, the processor 120 receives information for identifying third data on the user's physical condition through a first user input from the server 108. After displaying information for identifying the third data from the server 108 through the display 310, the processor 120 may identify the third data on the user's physical condition based on the first user input.

The processor 120 may display a first visual object for guiding a first activity within a second designated time interval 902, superimposed on a screen including time information. For example, in the second designated time interval 902, the first visual object is displayed, superimposed on a screen including time information. For example, the second designated time interval 902 is set to 3 days. After the second designated time interval 902 elapses, the processor 120 may remove the first visual object from the screen including time information.

The processor 120 may identify a second activity based on second data within a third designated time interval 903 including the second designated time interval 902. For example, in the third designated time interval 903, the processor 120 obtains the user's physical data and the user's psychological data included in the second data. For example, the duration of the third designated time interval 903 corresponds to the duration of the first designated time interval 901. The third designated time interval 903 may be set to 2 weeks.

According to an embodiment, after the third designated time interval 903 elapses, the processor 120 may identify data on the user's physical condition (hereinafter, referred to as fourth data) based on a user input (hereinafter, referred to as a second user input). For example, the processor 120 receives information for identifying fourth data on the user's physical condition through a second user input from the server 108. After displaying information for identifying the fourth data from the server 108 through the display 310, the processor 120 may identify the fourth data on the user's physical condition based on the second user input.

According to an embodiment, an operation similar to an operation of the processor 120 within the second designated time interval 902 may be performed within a fourth designated time interval 904. An operation similar to an operation of the processor 120 within the first designated time interval 901 or the third designated time interval 903 may be performed within a fifth designated time interval 905. According to an embodiment, the processor 120 may perform an operation according to the above-described embodiment until data (e.g., third data or fourth data) on the user's physical condition satisfies a designated condition.

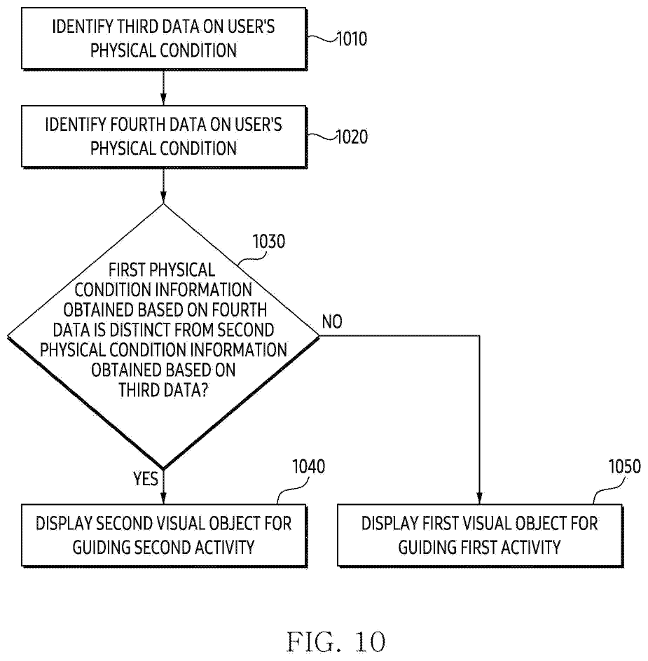

FIG. 10 is another flowchart illustrating an operation of an electronic device according to an embodiment of the disclosure.

This method may be executed by the electronic device 101 and the processor 120 of the electronic device 101 illustrated in FIGS. 2 and 3.

Referring to FIG. 10, operations 1010 to 1050 may be related to the operations 510 to 520 of FIG. 5. In operation 1010, the processor 120 may identify third data on the user's physical condition after the first designated time interval elapses. For example, after identifying the first data within the first designated time interval, the processor 120 identifies the third data on the user's physical condition.

For example, the processor 120 displays information for identifying the third data on the user's physical condition through a first user input, through the display 310. As an example, the information for identifying the third data on the user's physical condition may include a first content for a diagnostic and statistical manual of mental disorders (DSM)-5 level survey. The processor 120 may display the first content for the DSM-5 level survey through the display 310. The processor 120 may identify the first user input based on displaying the first content for the DSM-5 level survey. The processor 120 may identify third data on the user's physical condition related to mental illness based on the first user input.

According to an embodiment, after the first designated time interval elapses, the processor 120 may receive the third data on the user's physical condition from an external electronic device 202 connected with the electronic device 101 based on the first data. For example, the processor 120 transmits a signal for requesting the third data on the user's physical condition to the external electronic device 202, based on the first data. The external electronic device 202 may obtain the third data on the user's physical condition based on the user input. The external electronic device 202 may transmit the obtained third data on the user's physical condition to the electronic device 101. The processor 120 may receive the third data on the user's physical condition from the external electronic device 202.

In operation 1020, the processor 120 may identify fourth data on the user's physical condition. For example, after identifying the second data within the third designated time interval, the processor 120 identifies the fourth data on the user's physical condition. The processor 120 may identify the fourth data on the user's physical condition in the same as or similar to an operation for identifying the third data. For example, after the third designated time interval elapses, the processor 120 identifies the fourth data on the user's physical condition based on the second user input.

In operation 1030, the processor 120 may identify whether first physical condition information obtained based on the fourth data is distinct from second physical condition information obtained based on the third data. The processor 120 may identify whether the user's physical condition is changed by identifying whether the first physical condition information is distinct from the second physical condition information.

For example, the processor 120 identifies a first value related to the user's melancholy included in the first physical condition information, based on the fourth data. The processor 120 may identify a second value of the user's melancholy included in the second physical condition information, based on the third data. The processor 120 may identify whether the first value is distinct from the second value. The processor 120 may identify that the user's melancholy is changed based on the first value being distinct from the second value.

In operation 1040, when the first physical condition information is distinct from the second physical condition information, the processor 120 may display a second visual object for guiding a second activity. The processor 120 may display the second visual object for guiding the second activity based on the first physical condition information being distinct from the second physical condition information. Therefore, the processor 120 may perform operation 520 of FIG. 5 based on the first physical condition information being distinguished from the second physical condition information.

In operation 1050, when the first physical condition information is not distinct from the second physical condition information, the processor 120 may display the first visual object for guiding the first activity. The processor 120 may display the first visual object for guiding the first activity based on that the first physical condition information is not distinct from the second physical condition information. Therefore, the processor 120 may display the first visual object displayed within the second designated time interval even after the third designated time interval has elapsed, based on identifying that the first physical condition information has not changed compared to the second physical condition information.

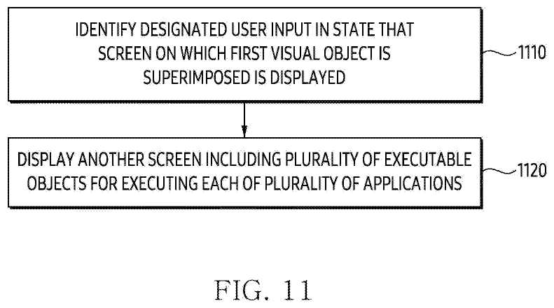

FIG. 11 is another flowchart illustrating an operation of an electronic device according to an embodiment of the disclosure.

This method may be executed by the electronic device 101 and the processor 120 of the electronic device 101 illustrated in FIGS. 2 and 3.

Referring to FIG. 11, in operation 1110, the processor 120 of the electronic device 101 may identify a designated user input in a state that a screen on which the first visual object is superimposed is displayed. For example, the processor 120 displays the first visual object, superimposed on a screen including time information. The processor 120 may identify the designated user input in a state in which the screen is displayed.

For example, the designated user input is variously set. The designated user input may be set to one of a swipe input, a tap input, a double tap input, and an input for a button of the electronic device 101.

In operation 1120, the processor 120 may display another screen including a plurality of executable objects for executing each of a plurality of applications. For example, a first executable object for executing a first application among the plurality of executable objects are highlighted relative to remaining executable objects among the plurality of executable objects. The processor 120 may display the first executable object for executing the first application in order to highlight the remaining executable objects among the plurality of executable objects.

For example, the processor 120 highlights the first executable object with respect to the remaining executable objects by displaying the first executable object along at least one edge of the first executable object as a visual element with a designated color. The first executable object may be highlighted with respect to the remaining executable objects, by being displayed as the visual element with a designated color along at least one edge of the first executable object.

As another example, the processor 120 may highlight the first executable object with respect to the remaining executable objects by displaying the first executable object to flicker. The first executable object may be highlighted with respect to the remaining executable objects, by being displayed to flicker.

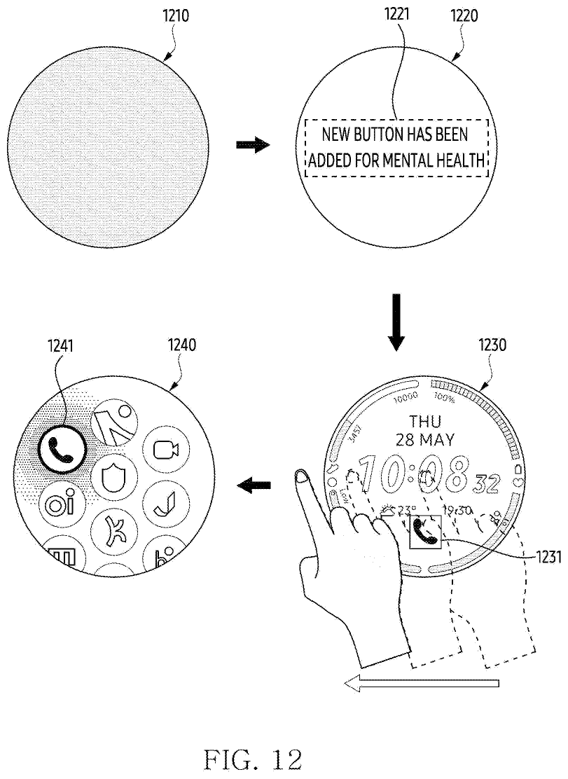

FIG. 12 illustrates another example of a screen displayed on an electronic device according to an embodiment of the disclosure.

Referring to FIG. 12, the processor 120 may display a screen 1210 in a state that the display 310 is inactivated. For example, the processor 120 displays a screen 1210 on which a black image is displayed through the inactivated display 310.

The processor 120 may identify a user input for switching a state of the display 310 from an inactive state to an active state. For example, the user input includes a double tap input or an input for a button of the electronic device 101. The processor 120 may switch the state of the display 310 from the inactive state to the active state in response to the user input. In response to detecting that the display 310 is switched from the inactive state to the active state, the processor 120 may display a notification 1221 for indicating that a first visual object is added through the screen 1220, instead of displaying a screen including time information.

The processor 120 may display a screen 1230 including time information after a certain time elapses from a timing at which the notification 1221 is displayed through the screen 1220. For example, the processor 120 switches the screen 1220 to the screen 1230 after a certain time elapses from a timing at which the notification 1221 is displayed. The processor 120 may display the first visual object 1231, superimposed on the screen 1230.

For example, the processor 120 identifies one of a first type to a fourth type, based on a first parameter (e.g., the first parameter of FIG. 7) and a second parameter (e.g., the second parameter of FIG. 7). The processor 120 may display a visual object (e.g., the first visual object 1231) for guiding one of a plurality of activities for the identified type. According to an embodiment, the processor 120 may identify that at least one of the first parameter and the second parameter is changed. The processor 120 may identify another activity distinct from the guided activity, based on identifying that at least one of the first parameter and the second parameter is changed. The processor 120 may change the displayed visual object (e.g., the first visual object 1231) to another visual object for guiding the other activity.

The processor 120 may identify a designated user input in a state that the screen 1230 is displayed. For example, the designated user input is a user input for switching to a screen including a plurality of executable objects for executing each of a plurality of applications. As an example, the designated user input may include a swipe input or an input for a button of the electronic device 101.

The processor 120 may switch the screen 1230 to a screen 1240 including the plurality of executable objects for executing each of the plurality of applications, in response to the designated user input.

For example, a first executable object 1241 for executing a first application among the plurality of executable objects are highlighted relative to remaining executable objects among the plurality of executable objects. The processor 120 may display the first executable object 1241 for executing the first application, in order to be highlighted relative to remaining executable objects among the plurality of executable objects. The processor 120 may display the first executable object 1241 to include a visual element having a designated color along at least one edge of the first executable object 1241. The processor 120 may highlight the first executable object 1241 with respect to the remaining executable objects, by displaying the first executable object 1241 to include the visual element.

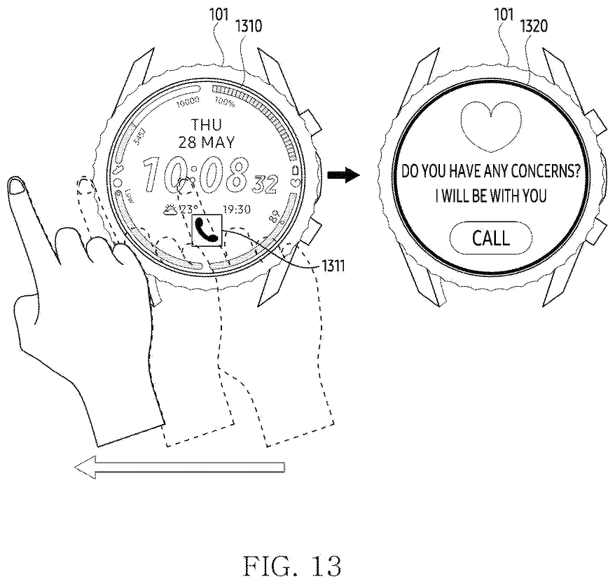

FIG. 13 illustrates still another example of a screen displayed on an electronic device according to an embodiment of the disclosure.

Referring to FIG. 13, the processor 120 may identify a designated user input, in a state that a first visual object 1311 usable for executing a first application is superimposed and displayed, and a screen 1310 including time information is displayed. For example, the designated user input is a user input for displaying one of a plurality of widgets capable of being displayed on the electronic device 101. Whenever the processor 120 identifies the designated user input, the processor 120 may sequentially display the plurality of widgets. For example, the designated user input includes a swipe input or an input for a button of the electronic device 101.

The processor 120 may switch the screen 1310 to a screen 1320 in response to a designated user input. The processor 120 may first display a widget for the first application based on a designated user input. The processor 120 may display the widget for the first application on the screen 1320. The processor 120 may display a widget for the first application to include a visual element having a designated color along at least one edge of the widget for the first application.

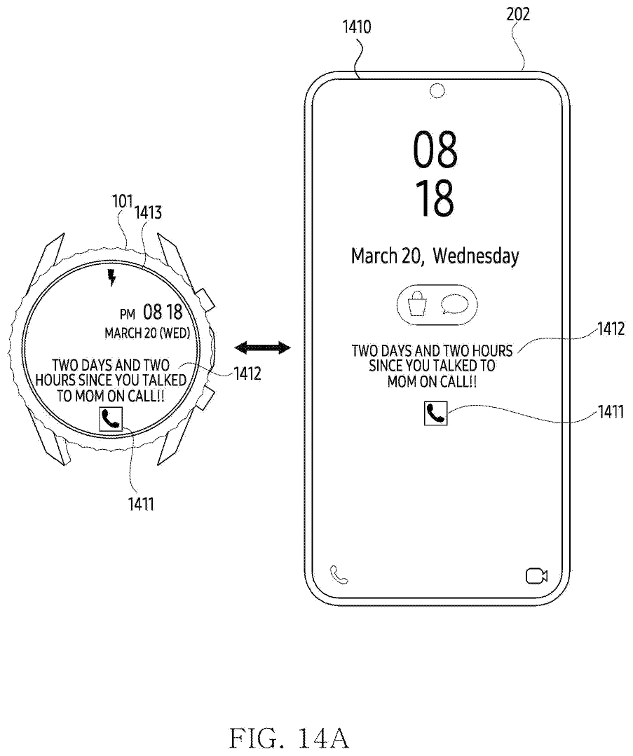

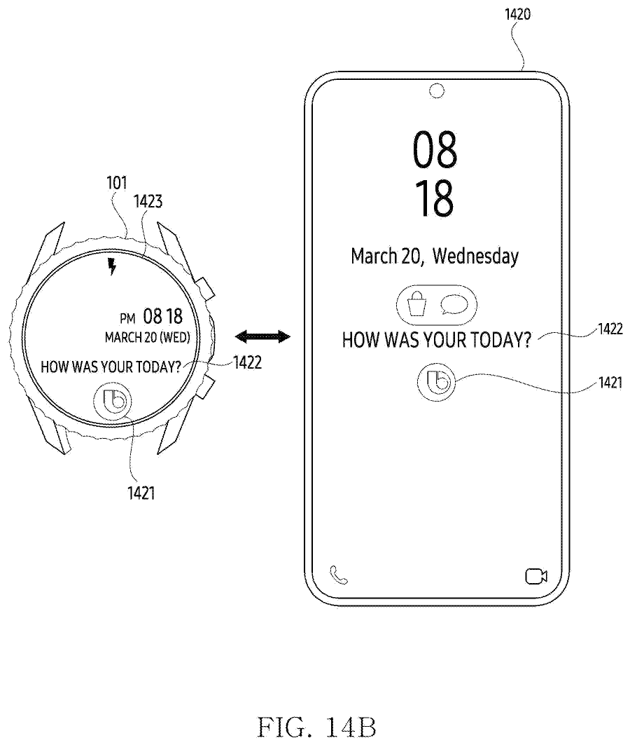

FIG. 14A illustrates an example of a screen displayed on an electronic device and an external electronic device according to an embodiment of the disclosure.

FIG. 14B illustrates another example of a screen displayed on an electronic device and an external electronic device according to an embodiment of the disclosure.

Referring to FIGS. 14A and 14B, the processor 120 may transmit a signal to request that the external electronic device 202 connected with the electronic device 101 displays a first visual object (e.g., a visual object 1411 or a visual object 1421) to the external electronic device 202. The visual object may be displayed, superimposed on a lock screen within a second designated time interval of the external electronic device 202.

Referring to FIG. 14A, the processor 120 may transmit a signal to request that the external electronic device 202 display a visual object 1411 for guiding a phone call to the external electronic device 202. Based on the signal, the external electronic device may display the visual object 1411, superimposed on the lock screen 1410. The processor 120 may display a visual object 1412 including text for suggesting use of a phone call application related to a phone call, superimposed on the lock screen 1410 together with the visual object 1411.

According to an embodiment, the visual object 1411 and the visual object 1412 may be displayed not only on the lock screen 1410 displayed on the external electronic device 202, but also on the display 310 of the electronic device 101, and a screen 1413 including time information.

Referring to FIG. 14B, the processor 120 may transmit a signal to request that the external electronic device 202 display a visual object 1421 for activating a conversation through artificial intelligence to the external electronic device 202. The external electronic device may display the visual object 1421 superimposed on a lock screen 1420, based on the signal. The processor 120 may display a visual object 1422 including text for suggesting a conversation through artificial intelligence, superimposed on the lock screen 1420 together with the visual object 1421.

According to an embodiment, the visual object 1421 and the visual object 1422 may be displayed not only on the lock screen 1420 displayed on the external electronic device 202, but also on the display 310 of the electronic device 101, and a screen 1423 including time information.

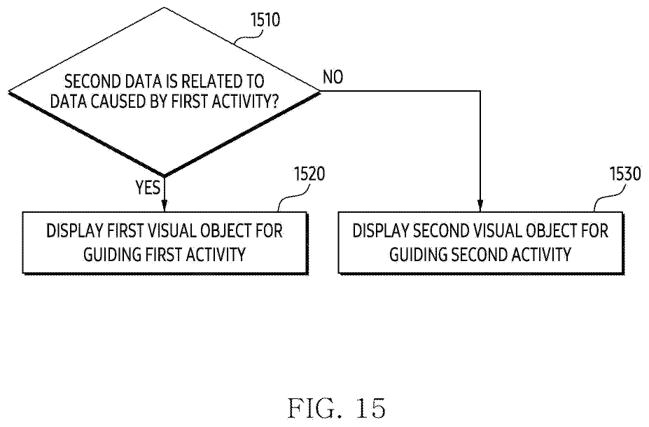

FIG. 15 is another flowchart illustrating an operation of an electronic device according to an embodiment of the disclosure.

This method may be executed by the electronic device 101 and the processor 120 of the electronic device 101 illustrated in FIGS. 2 and 3.

Referring to FIG. 15, operations 1510 to 1530 may be related to operations 510 to 520 of FIG. 5. In operation 1510, the processor 120 may identify whether second data is related to data caused by a first activity. For example, the processor 120 identifies that a user input for a first visual object is received. The processor 120 may identify whether the first activity is performed by the user through execution of the first application. The processor 120 may identify whether the first activity is performed by the user by identifying whether the second data is related to the data caused by the first activity.

In operation 1520, when the second data is related to the data caused by the first activity, the processor 120 may display the first visual object for guiding the first activity. The processor 120 may display the first visual object for guiding the first activity based on that the second data is related to the data caused by the first activity. After the first visual object is displayed, the processor 120 may display the first visual object for guiding the same first activity, superimposed on a screen including time information, based on identifying that the first activity is performed by the user.

In operation 1530, when the second data is not related to the data caused by the first activity, the processor 120 may display a second visual object for guiding a second activity. The processor 120 may display the second visual object for guiding the second activity, based on that the second data is not related to the data caused by the first activity. After the first visual object is displayed, the processor 120 may display the second visual object for guiding the second activity distinct from the first activity, superimposed on a screen including time information, based on identifying that the first activity is not performed by the user.

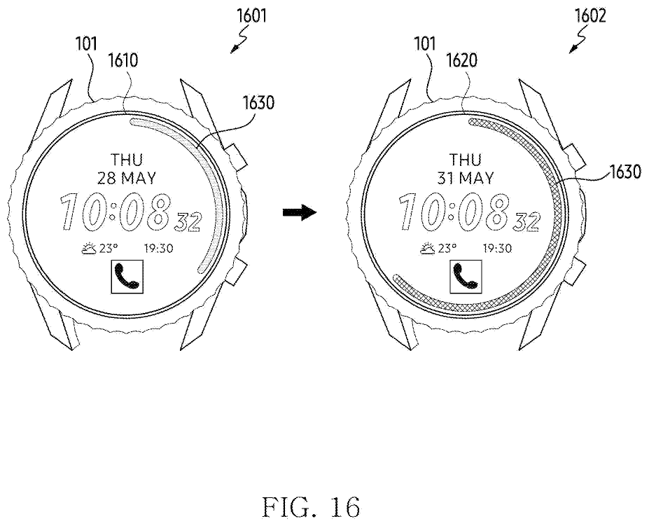

FIG. 16 illustrates still another example of a screen displayed on an electronic device according to an embodiment of the disclosure.

Referring to FIG. 16, the processor 120 of the electronic device 101 may display a bar-shaped indicator 1630 of which a length is changed based on that a designated time interval (e.g., the third designated time interval of FIG. 5) elapses on a screen 1610. For example, the processor 120 changes the length of the indicator 1630 as the designated time interval elapses. The length of the indicator 1630 may increase along an edge of the screen 1610 as the designated time interval elapses.

According to an embodiment, the processor 120 may identify whether the length of the indicator 1630 is greater than or equal to a designated length. The processor 120 may display a color of the indicator as a designated color based on identifying that the length of the indicator 1630 is greater than or equal to the designated length (e.g., half of the circumference of the screen 1610).

For example, in a state 1601, the processor 120 identifies that the length of the indicator 1630 is less than the designated length (e.g., half of the circumference of the screen 1610). The processor 120 may display a color of the indicator 1630 as a first color. As the designated time elapses, the length of the indicator 1630 may be changed. In a state 1602, the processor 120 may identify that the length of the indicator 1630 is greater than or equal to the designated length (e.g., half of the circumference of the screen 1620). The processor 120 may display the color of the indicator 1630 as a second color based on identifying that the length of the indicator 1630 is greater than or equal to the designated length. The processor 120 may change the color of the indicator 1630 from the first color to the second color, based on identifying that the length of the indicator 1630 is greater than or equal to the designated length.

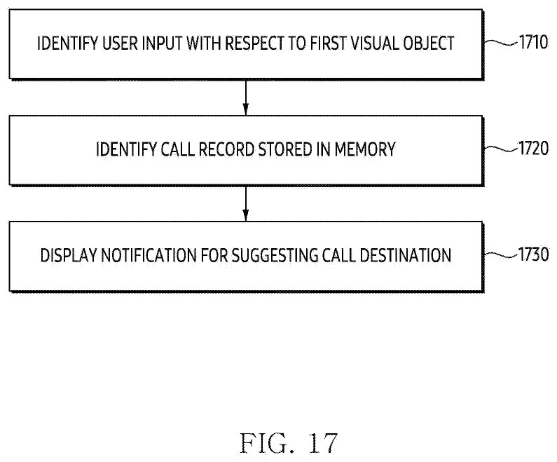

FIG. 17 is still another flowchart illustrating an operation of an electronic device according to an embodiment of the disclosure.

This method may be executed by the electronic device 101 and the processor 120 of the electronic device 101 illustrated in FIGS. 2 and 3.

Referring to FIG. 17, in operation 1710, the processor 120 of the electronic device 101 may identify a user input with respect to a first visual object. For example, the processor 120 identifies a user input for the first visual object usable for executing an application for a phone call.

In operation 1720, the processor 120 may identify a call record stored in the memory 130. For example, the processor 120 identifies a call destination calling for a certain time or longer from among a plurality of call destinations, based on a call record. For another example, the processor 120 may identify a call destination calling at a designated time (e.g., dawn) or a designated day (e.g., weekend) among the plurality of call destinations.

In operation 1730, the processor 120 may display a notification for suggesting a call destination on the display 310, based on the call record. For example, the processor 120 displays the notification for suggesting a call destination, superimposed on a screen including time information.

For example, the processor 120 identifies a call destination calling for a certain time or longer among the plurality of call destinations, and designate the identified call destination as a call destination being proposed through the notification. For another example, the processor 120 may designate a call destination calling on a designated day (e.g., weekend) among the plurality of call destinations as a call destination being proposed through the notification.

Unlike the above-described embodiment, according to an embodiment, instead of displaying the notification for suggesting a call destination, the processor 120 may perform a call connection with the person who has called the most in response to a user input for the first visual object.

According to various embodiments, an electronic device (e.g., the electronic device 101 of FIG. 3) may comprise a display (e.g., the display 301 of FIG. 3), at least one sensor (e.g., the processor 120 of FIG. 3), memory (e.g., the memory 130 of FIG. 3) storing one or more computer programs, and one or more processors communicatively coupled to the display, the at least one sensor (e.g., the at least one sensor 330 of FIG. 3) and the memory, wherein the one or more computer programs include computer-executable instructions that, when executed by the one or more processors, cause the electronic device to display a first visual object for guiding a first activity identified based on first data obtained from the electronic device in a first designated time interval, superimposed on a screen including time information in a second designated time interval, and after displaying the first visual object in the second designated time interval, display a second visual object for guiding a second activity distinct from the first activity, identified based on second data obtained in a third designated time interval including the second designated time interval, superimposed on the screen, wherein the first visual object may be usable for executing a first application related to the first activity, and wherein the second visual object is usable for executing a second application related to the second activity, distinct from the first application.

According to an embodiment, the electronic device may further comprise a communication circuit coupled to the one or more processors, wherein the one or more computer programs further include computer-executable instructions that, when executed by the one or more processors, cause the electronic device to, after the first designated time interval elapses, receive, based on the first data, third data related to a body condition of a user of the electronic device obtained from an external electronic device connected with the electronic device.

According to an embodiment, wherein the at least one processor may be further configured to, after the first designated time interval elapses, identify, based on a first user input, third data related to a body condition of a user of the electronic device.

According to an embodiment, the one or more computer programs further include computer-executable instructions that, when executed by the one or more processors, cause the electronic device to, after the third designated time interval elapses, identify, based on a second user input, fourth data related to the body condition of the user.

According to an embodiment, the at least one processor may be configured to identify that first body condition information of the user obtained based on the fourth data is distinct from second body condition information obtained based on the third data, and in response to identifying that the first body condition information is distinct from the second body condition information, display, based on the second data, the second visual object for guiding the second activity.

According to an embodiment, the first data may comprise physical data of the user and psychological data of the user.

According to an embodiment, the one or more computer programs further include computer-executable instructions that, when executed by the one or more processors, cause the electronic device to identify at least one of data related to a movement of the user, data related to walking of the user, and data related to a first sleep state of the user as the physical data of the user, and identify at least one of data related to a stress level of the user and data related to a second sleep state as the psychological data of the user.

According to an embodiment, the one or more computer programs further include computer-executable instructions that, when executed by the one or more processors, cause the electronic device to identify, based on the physical data of the user and the psychological data of the user, one of a first type to a fourth type, and identify one of a plurality of activities related to the identified type as the first activity.

According to an embodiment, the electronic device may further comprise a communication circuit communicatively coupled to the one or more processor, wherein the one or more computer programs further include computer-executable instructions that, when executed by the one or more processors, cause the electronic device to identify a user input on the first visual object, and in response to the user input, transmit a signal for executing the first application to an external electronic device connected with the electronic device, and wherein the first application may be executed in the external electronic device based on the signal.

According to an embodiment, the one or more computer programs further include computer-executable instructions that, when executed by the one or more processors, cause the electronic device to identify a designated user input in a state where the screen on which the first visual object is superimposed is displayed, and in response to the designated user input, display another screen comprising a plurality of executable objects for executing a plurality of application respectively, switched from the screen, and wherein a first executable object for executing the first application among the plurality of executable objects may be highlighted relative to remaining executable objects among the plurality of executable objects.

According to an embodiment, the first executable object may be highlighted relative to the remaining executable objects among the plurality of executable objects by displaying a visual element having a designated color along at least one edge of the first executable object.

According to an embodiment, the one or more computer programs further include computer-executable instructions that, when executed by the one or more processors, cause the electronic device to, in response to detecting that a state of the display is switched from an inactivated state to an activated state after identifying the first activity based on the first data, display a screen comprising a notification to indicate that the first visual object has been added instead of displaying the screen.

According to an embodiment, the first application may comprise an application for phone calls, wherein among the plurality of executable objects to identify a user input on the first visual object which is usable for executing the first application, identify, based on the user input on the first visual object, a call record stored in the memory, and display, based on the call record, a notification for suggesting a call destination.

According to an embodiment, the one or more computer programs further include computer-executable instructions that, when executed by the one or more processors, cause the electronic device to identify a call destination calling a time greater than or equal to a designated time among a plurality of call destinations based on the call record, and designate the identified call destination as the call destination being proposed through the notification.

According to an embodiment, the first visual object superimposed and displayed on the screen may be displayed within the second designated time interval by a designated cycle.

According to an embodiment, the one or more computer programs further include computer-executable instructions that, when executed by the one or more processors, cause the electronic device to display a third visual object including text for proposing use of the first application, superimposed on the screen together with the first visual object.

According to an embodiment, the electronic device may further comprise a communication circuit communicatively coupled to the electronic device, wherein the one or more computer programs further include computer-executable instructions that, when executed by the one or more processors, cause the electronic device to transmit a signal to the external electronic device to request that the first visual object is displayed in an external electronic device connected to the electronic device, and wherein the first visual object may be displayed superimposed on a lock screen of the external electronic device within the second designated time interval.

According to an embodiment, the one or more computer programs further include computer-executable instructions that, when executed by the one or more processors, cause the electronic device to identify whether the second data obtained within the third designated time interval is related to data caused by the first activity, and display the second visual object, superimposed on the screen, based on identifying that the second data is not related to the data caused by the first activity.

According to an embodiment, the one or more computer programs further include computer-executable instructions that, when executed by the one or more processors, cause the electronic device to display a bar-shaped indicator of which a length is changed based on that the third designated time interval elapses along an edge of the screen, identify whether a length of the indicator is greater than or equal to a designated length, and display a color of the bar-shaped indicator in a designated color, based on identifying that the length of the indicator is greater than or equal to the designated length.

According to various embodiments, a method performed by an electronic device is provided. The method may include displaying a first visual object for guiding a first activity identified based on first data obtained from the electronic device in a first designated time interval, superimposed on a screen including time information in a second designated time interval, and after displaying the first visual object in the second designated time interval, displaying a second visual object for guiding a second activity distinct from the first activity, identified based on second data obtained in a third designated time interval including the second designated time interval, superimposed on the screen, wherein the first visual object may be usable for executing a first application related to the first activity, and wherein the second visual object may be usable for executing a second application related to the second activity, distinct from the first application.

According to various embodiments, one or more non-transitory computer-readable storage media may store one or more programs comprising computer-executable instructions that, when being executed by one or more processors of an electronic device including a display and at least one sensor, cause the electronic device to perform operations, the operations including displaying, by the electronic device, a first visual object for guiding a first activity identified based on first data obtained from the electronic device in a first designated time interval, superimposed on a screen including time information in a second designated time interval, and after displaying the first visual object in the second designated time interval, displaying, by the electronic device, a second visual object for guiding a second activity distinct from the first activity, identified based on second data obtained in a third designated time interval including the second designated time interval, superimposed on the screen, wherein the first visual object may be usable for executing a first application related to the first activity, and wherein the second visual object may be usable for executing a second application related to the second activity, distinct from the first application.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” or “connected with” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module is implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) invokes at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between a case in which data is semi-permanently stored in the storage medium and a case in which the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a display;
at least one sensor;
memory storing one or more computer programs; and
one or more processors communicatively coupled to the display, the at least one sensor and the memory,
wherein the one or more computer programs include computer-executable instructions that, when executed by the one or more processors, cause the electronic device to:
display a first visual object for guiding a first activity identified based on first data obtained from the electronic device in a first designated time interval, superimposed on a screen including time information in a second designated time interval, and
after displaying the first visual object in the second designated time interval, display a second visual object for guiding a second activity distinct from the first activity, identified based on second data obtained in a third designated time interval including the second designated time interval, superimposed on the screen,
wherein the first visual object is usable for executing a first application related to the first activity, and
wherein the second visual object is usable for executing a second application related to the second activity, distinct from the first application.

2. The electronic device of claim 1, further comprising:
a communication circuit coupled to the one or more processors,
wherein the one or more computer programs further include computer-executable instructions that, when executed by the one or more processors, cause the electronic device to:
after the first designated time interval elapses, receive, based on the first data, third data related to a body condition of a user of the electronic device obtained from an external electronic device connected with the electronic device.

3. The electronic device of claim 1, wherein the one or more computer programs further include computer-executable instructions that, when executed by the one or more processors, cause the electronic device to, after the first designated time interval elapses, identify, based on a first user input, third data related to a body condition of a user of the electronic device.

4. The electronic device of claim 3, wherein the one or more computer programs further include computer-executable instructions that, when executed by the one or more processors, cause the electronic device to, after the third designated time interval elapses, identify, based on a second user input, fourth data related to the body condition of the user.

5. The electronic device of claim 4, wherein the one or more computer programs further include computer-executable instructions that, when executed by the one or more processors, cause the electronic device to:
identify that first body condition information of the user obtained based on the fourth data is distinct from second body condition information obtained based on the third data, and
in response to identifying that the first body condition information is distinct from the second body condition information, display, based on the second data, the second visual object for guiding the second activity.

6. The electronic device of claim 1, wherein the first data comprises physical data of a user of the electronic device and psychological data of the user.

7. The electronic device of claim 6, wherein the one or more computer programs further include computer-executable instructions that, when executed by the one or more processors, cause the electronic device to:
identify at least one of data related to a movement of the user, data related to walking of the user, and data related to a first sleep state of the user as the physical data of the user, and
identify at least one of data related to a stress level of the user and data related to a second sleep state as the psychological data of the user.

8. The electronic device of claim 7, wherein the one or more computer programs further include computer-executable instructions that, when executed by the one or more processors, cause the electronic device to:
identify, based on the physical data of the user and the psychological data of the user, one of a first type to a fourth type, and
identify one of a plurality of activities related to the identified type as the first activity.

9. The electronic device of claim 1, further comprising:
a communication circuit coupled to the one or more processors,
wherein the one or more computer programs further include computer-executable instructions that, when executed by the one or more processors, cause the electronic device to:
identify a user input on the first visual object, and
in response to the user input, transmit a signal for executing the first application to an external electronic device connected with the electronic device,
wherein the first application is executed in the external electronic device based on the signal.

10. The electronic device of claim 1,
wherein the one or more computer programs further include computer-executable instructions that, when executed by the one or more processors, cause the electronic device to:
identify a designated user input in a state where the screen on which the first visual object is superimposed is displayed, and
in response to the designated user input, display another screen comprising a plurality of executable objects for executing a plurality of application respectively, switched from the screen, and
wherein a first executable object for executing the first application among the plurality of executable objects is highlighted relative to remaining executable objects among the plurality of executable objects.

11. The electronic device of claim 10, wherein the first executable object is highlighted relative to the remaining executable objects among the plurality of executable objects by displaying a visual element having a designated color along at least one edge of the first executable object.

12. The electronic device of claim 1, wherein the one or more computer programs further include computer-executable instructions that, when executed by the one or more processors, cause the electronic device to:
in response to detecting that a state of the display is switched from an inactivated state to an activated state after identifying the first activity based on the first data, display a screen comprising a notification to indicate that the first visual object has been added instead of displaying the screen.

13. The electronic device of claim 1,
wherein the first application comprises an application for phone calls,
wherein the one or more computer programs further include computer-executable instructions that, when executed by the one or more processors, cause the electronic device to:
identify a user input on the first visual object which is usable for executing the first application,
identify, based on the user input on the first visual object, a call record stored in the memory, and
display, based on the call record, a notification for suggesting a call destination.

14. A method performed by an electronic device comprising:
displaying, by the electronic device, a first visual object for guiding a first activity identified based on first data obtained from the electronic device in a first designated time interval, superimposed on a screen including time information in a second designated time interval; and
after displaying the first visual object in the second designated time interval, displaying, by the electronic device, a second visual object for guiding a second activity distinct from the first activity, identified based on second data obtained in a third designated time interval including the second designated time interval, superimposed on the screen,
wherein the first visual object is usable for executing a first application related to the first activity, and
wherein the second visual object is usable for executing a second application related to the second activity, distinct from the first application.

15. The method of claim 14, further comprising:
after the first designated time interval elapses, receiving, based on the first data, third data related to a body condition of a user of the electronic device obtained from an external electronic device connected with the electronic device.

16. The method of claim 14, further comprising:
after the first designated time interval elapses, identifying, based on a first user input, third data related to a body condition of a user of the electronic device.

17. The method of claim 16, further comprising:
after the third designated time interval elapses, identifying, based on a second user input, fourth data related to the body condition of the user.

18. The method of claim 17, further comprising:
identifying that first body condition information of the user obtained based on the fourth data is distinct from second body condition information obtained based on the third data; and
in response to identifying that the first body condition information is distinct from the second body condition information, displaying, based on the second data, the second visual object for guiding the second activity.

19. One or more non-transitory computer-readable storage media storing one or more programs including computer-executable instructions that, when executed by one or more processors of an electronic device including a display and at least one sensor, cause the electronic device to perform operations, the operations comprising:
displaying, by the electronic device, a first visual object for guiding a first activity identified based on first data obtained from the electronic device in a first designated time interval, superimposed on a screen including time information in a second designated time interval, and
after displaying the first visual object in the second designated time interval, displaying, by the electronic device, a second visual object for guiding a second activity distinct from the first activity, identified based on second data obtained in a third designated time interval including the second designated time interval, superimposed on the screen,
wherein the first visual object is usable for executing a first application related to the first activity, and
wherein the second visual object is usable for executing a second application related to the second activity, distinct from the first application.

20. The one or more non-transitory computer-readable storage media of claim 19, the operations further comprising:
after the first designated time interval elapses, receiving, based on the first data, third data related to a body condition of a user of the electronic device obtained from an external electronic device connected with the electronic device.
